(19) United States
(12) Patent Application Publication
(10) Pub. No.: US 2002/0103181 A1
(43) Pub. Date: Aug. 1, 2002

(54) Pharmaceutical composition for controlled release of an active ingredient

(75) Inventors: Himadri Sen, Maharashtra (IN); Rajesh Suresh Kshirsagar, Maharashtra (IN); Chandrashekhar Shriram Kandi, Maharashtra (IN); Shailesh Suresh Bhamare, Maharashtra (IN)

Correspondence Address: Ladas & Parry, 26 West 61st Street, New York, NY, 10023, US

(73) Assignee: LUPIN LABORATORIES LIMITED

(21) Appl. No.: 09/726,636

(22) Filed: Nov. 30, 2000

(51) Int. Cl. A61K 31/545

(52) U.S. Cl. 514/200; 424/468

(57) ABSTRACT

A pharmaceutical composition in the form of a tablet for controlled release of an active ingredient comprises a betalactam antibiotic such as cephalexin, cefaclor or their pharmaceutically acceptable hydrates, salts or esters as active ingredient, and a mixture of hydrophilic polymers selected from the group consisting of at least one sodium alginate and at least one xanthan gum as controlled release matrix; and optionally probenecid as an antibiotic adjuvant as either immediate release or controlled release part. The composition may also contain one or more of a water soluble and/or water dispersible diluent, wherein the quantities of the hydrophilic polymers and water soluble and/or water dispersible diluents are such that the therapeutically effective active ingredient is released at a rate suitable for once or twice daily administration of the pharmaceutical composition. Inclusion of probenecid allows reduction in the amount of active incorporated in the hydrophilic polymer matrix but still provides the desired once a day profile. The resulting modified release matrix formulation not containing probenecid may be administered once or twice daily. The resulting modified release matrix formulation containing probenecid may be administered once daily.

### FIELD OF THE INVENTION

This invention relates to a pharmaceutical composition of modified release tablets comprising a betalactam antibiotic or their pharmaceutically acceptable hydrates, salts or esters as the active ingredient, and a mixture of hydrophilic polymers selected from the group consisting of at least one sodium alginate and at least one xanthan gum as controlled release matrix. More particularly the invention relates to pharmaceutical composition of modified release tablets in which the active material is selected from cephalexin, cefaclor or their pharmaceutically acceptable hydrates, salts or esters. The composition optionally comprises probenecid as an antibiotic adjuvant as either immediate release or controlled release part. Further optionally, the composition contains one or more of a water soluble and/or water dispersible diluent, wherein the quantities of the hydrophilic polymers and water soluble and/or water dispersible diluents are such that the therapeutically effective active ingredient is released at a rate suitable for once or twice daily administration of the pharmaceutical composition. Inclusion of probenecid allows reduction in the amount of active incorporated in the hydrophilic polymer matrix but still provides the desired once a day profile.

### BACKGROUND OF THE INVENTION

Most drugs used to treat microbial infections are given more than once during a dosage regimen. The objectives during antimicrobial therapy are to maximize blood concentration, preferably several fold higher than the minimum inhibitory concentration (MIC) for the particular agent, but to minimize both the risk of toxicity to the patient and of promoting microbial resistance. Although oral administration will be the preferred route, in the case of antibiotics this route is frequently unattractive because of their low or variable oral bioavailability. In addition extremely high plasma concentrations of antibiotics are frequently required to achieve their MIC values towards certain gram-negative bacteria. (Antibiotic and Chemotherapy: Anti-infective agents and their use in therapy, 7^{th} edition, Ed. by O'grady F., Finch R. G., Lambert H. P., Greenwood D.; Churchill Livingstone, 1997).

While many compounds are known to be useful as pharmacologically active substances, some of them have relatively short biological half-life and need to be administered several times a day in order to achieve desired therapeutic effect. However, a decrease in the frequency of administration will not only reduce the burden on the patient but will also increase compliance and thus provide greater therapeutic effect. It can be achieved by controlling the release of active ingredients, so that the effective level is maintained in the blood for a prolonged period of time or by reducing the elimination of the active from the body thereby increasing its concentration in blood resulting in its retention in blood for longer period.

This has been primarily achieved by development of new drug delivery systems utilizing diverse techniques and principles. Amongst these, known in the art is one such delivery system which employs hydrophilic polymers to produce sustained or modified release pharmaceutical compositions. For modified release solid dosage forms comprising a drug dispersed uniformly in hydrophilic polymers, release of the drug is controlled primarily by diffusion of the drug, or by surface erosion of the hydrophilic polymers into the surrounding medium, or by a combination of the two processes. Control of the rate of release can produce constant blood levels of the active ingredient that may result in reducing the frequency of administration, thereby improving patient compliance to the dosage regimen.

Many drugs and drug metabolites are actively secreted by the proximal tubular active transport mechanism and interactions may arise from competition for these systems. Particularly with antibiotic therapy, active tubular secretion is a significant route of elimination. Drugs that use the same active transport system in the kidney tubules can compete with one another for secretion. Probenecid belongs to this class of drugs, which is able to compete successfully with some other drugs for an active secretion mechanism in the kidney tubule. This prevents them from being secreted into the tubular filtrate. Probenecid is later passively reabsorbed from the kidney tubules. Probenecid was extremely useful in the early days of penicillin when the combination raised and prolonged penicillin plasma levels. Inhibition of the urinary excretion of penicillin and some cephalosporins has been used as a device to increase the biliary excretion of these agents, thereby raising the antibiotic concentrations in the biliary tract. This has been used to improve the efficacy of antibiotic treatment (Antibiotic and Chemotherapy: Anti-infective agents and their use in therapy, 7^{th} edition, Ed. by O'grady F., Finch R. G., Lambert H. P., Greenwood D.; Churchill Livingstone, 1997).

The relevant prior art methods, which teach adaptation of diverse delivery systems for sustained release of the active, are as follows.

U.S. Pat. No. 4,250,166 discloses a long-acting cephalexin preparation comprising of normal quick-releasing cephalexin and particulate cephalexin coated with a copolymer of methylmethacrylate and methacrylic acid which dissolves at a pH from 5.5 to 6.5 and the potency ratio of the normal cephalexin to coated cephalexin is between 40:60 and 25:75.

U.S. Pat. No. 4,713,247 discloses a long-acting cefaclor formulation comprising of a mixture of non-enteric coated rapid-release cefaclor component and an enteric coated slow-release cefaclor component at a ratio of 4:6 based upon cefaclor potency, wherein the rapid-release component releases the drug in gastric fluid while the slow-release component dissolves at pH 5 to 7, thereby enabling oral administration thereof twice a day.

U.S. Pat. No. 4,968,508 discloses a sustained release matrix tablet comprising from about 0.1% to about 90% by weight of cefaclor, about 5% to about 29% by weight of hydrophilic polymer and about 0.5% to about 25% by weight of an acrylic polymer which dissolves at a pH in the range of about 5.0 to about 7.4, the total weight of polymers being less than 30% by weight of the formulation. Although a specific cefaclor formulation is claimed, the text suggests that the matrix formulation is suitable for weakly basic drugs and particularly suitable for cephalexin and cefaclor.

U.S. Pat. No. 5,948,440 discloses a controlled release tablet of an active ingredient comprising of cefaclor, cephalexin, or their pharmaceutically acceptable hydrates, salts, or esters as active ingredient, and a mixture of hydrophilic polymers selected from the group consisting of at least one hydroxypropyl methylcellulose and at least one hydroxypropylcellulose. The composition optionally also contains one or more of a water soluble or water dispersible diluent. The quantities of the hydrophilic polymers and water soluble or water dispersible diluent are such that the therapeutically effective active ingredient is released at a rate suitable for twice daily administration of the pharmaceutical composition.

Patent Application WO 99/49868 discloses a sustained release cefaclor composition comprising 30 to 90 wt % of cefaclor, 5 to 60 wt % of a hydroswelling polymer and 1 to 10 wt % of a salt capable of releasing gaseous CO₂ in a gastric environment useful for administration once a day as well as twice a day. The amount of the salt added is critical as use of excessive amount of salt would generate excessive amount of CO₂ gas thereby irritating the stomach, disintegrating the formulation and losing the sustained release characteristic.

Japanese Patent JP 57165392A discloses a long-acting cephalexin tablet comprising cephalexin mixed with ≧10% w/w oils and fats (e.g. higher fatty acid, higher alcohol, alcohol ester, etc.) and with a vehicle such as microcrystalline cellulose and a lubricant such as magnesium stearate, and the mixture is pressed, formed to granules passing through a 20 mesh sieve, and subjected to the slug-forming process to obtain a high-quality long-acting tablet. The rate of dissolution of cephalexin can be controlled by selecting the kind of oils and fats and the number of the times of slug formation process.

Japanese Patent JP 07010758A discloses a long acting cefaclor composition comprising rapidly soluble cefaclor and a delayed soluble cefaclor prepared by enteric coating of hydroxypropyl methyl cellulose acetate succinate and triethyl citrate.

Patent application WO 98/22091 discloses a controlled release ss-lactam antibiotic agent preferably amoxicillin trihydrate in a hydrophilic and/or hydrophobic polymeric matrix such that 50% of the active is released within 3 to 4 hr from oral administration and remainder is released at a controlled rate. Examples include matrix tablets containing amoxicillin with hydroxypropyl methylcelluloses, amoxicillin with eudragit and alginate.

U.S. Pat. No. 3,996,355 teaches permanent suspension dosage forms of water-sensitive drugs for administration without reconstitution. Amoxicillin-probenecid suspension dispersed in sesame oil containing sucrose as suspending agent and silica as thickening agent is exemplified.

Patent No. RO 80932 discloses oral suspension of benzathine penicillin, procaine penicillin and probenecid with other excipients.

Japanese Patent JP 52105220A discloses suppository formulations of betalactams. For example, a suppository capsule containing cephalexin, probenecid, peanut oil and polyoxyethylene cetyl ether.

Japanese Patent JP 52064418A discloses highly absorbable penicillin suppository formulation containing (4-ethyl-2,3-dioxo-1-piperazinyl carbonyl amino)-benzyl penicillin or its salt, probenecid, and peanut oil.

U.S. Pat. No. 6,083,532 discloses a sustained release tablet comprising a drug to be released at a controlled rate and a sustained release formulation comprising at least three different types of polymers including a pH dependent gelling polymer, a pH independent gelling polymer and an enteric polymer wherein pH dependent gelling polymer comprises at least one of an alginate, a carboxyvinyl polymer, or a salt of a carboxymethyl cellulose; pH independent gelling polymer comprises at least one of a hydroxy propyl methyl cellulose, a hydroxy propyl ethyl cellulose, a hydroxy propyl cellulose, a hydroxy ethyl cellulose, a methyl cellulose, a xantham gum or a polyethylene oxide; and enteric polymer comprises at least one of a polyacrylate material, a cellulose acetate phthalate, a cellulose phthalate hydroxy propyl methyl ether, a polyvinyl acetate phthalate, a hydroxy propyl methyl cellulose acetate succinate, a cellulose acetate trimellitate, or a shellac.

Patent application WO 00/15198 teaches controlled delivery pharmaceutical composition having temporal and spatial control, comprising a drug, a gas generating component, a swelling agent, a viscolyzing agent, and optionally a gel forming polymer. The viscolyzing agent initially and the gel forming polymer thereafter form a hydrated gel matrix which entraps the gas, causing the tablet to float so that it is retained in the stomach thereby providing spatial control and at the same time resulting in sustained release of the drug providing temporal control. The combination of gas generating component, swelling agent and viscolyzing agent results in the controlled drug delivery system. Thus all these components are essential for achieving the temporal and spatial control. A preferred once daily ciprofloxacin formulation comprising 69.9% ciprofloxacin base, 0.34% sodium alginate, 1.03% xanthan gum, 13.7% sodium bicarbonate, 12.1% cross-linked polyvinylpyrrolidone and optionally other excipients is disclosed.

There exists a need for a pharmaceutical composition that can provide controlled release of cephalexin or cefaclor such that it is maintained in the blood at therapeutically effective level for 24 hr resulting in once-daily administration of the composition thereby improving patient compliance to the dosage regimen. This may be achieved by two interventions:

1. In-vitro—Controlling the release profile of the active ingredient from the delivery system, which in turn, can prolong blood levels over extended period of time.

2. In-vivo—Using probenecid as an adjuvant thereby reducing the elimination/excretion of the active ingredient from the body.

The first intervention alone may result in extended release delivery system for active suitable for once or twice daily administration of the composition depending on the composition of the controlled release matrix. The combination of these two interventions can afford extended release antibiotic composition suitable for once daily administration.

The excretion of the betalactam antibiotics which are excreted by renal tubular secretion is reduced when given concurrently with probenecid resulting in increased and prolonged antibiotic serum concentration and prolonged half life.

The present invention gives the advantage of administering the drug in a manner that it achieves once a day profile either through only in-vitro intervention i.e. by controlling the release profile of the active ingredient from the delivery system, wherein the amount of the active incorporated in the hydrophilic polymer matrix is higher; or through combination of in-vitro and in-vivo interventions, wherein owing to probenecid the amount of active incorporated in the hydrophilic polymer matrix can be reduced but still achieving the desired once a day profile.

Since the antibiotics are high dosing/high frequency, extended release drug delivery systems have not been very successful in reducing the frequency of dosing. The in-vitro intervention where the release profile of the active ingredient from the delivery system is controlled by the specific polymers present in the matrix and their concentration thereby allowing prolonged blood levels over extended period of time, is employed to achieve long acting cephalexin or cefaclor compositions that may be administered once daily or twice daily. Whereas combination of in-vitro and in-vivo interventions where a combination of extended release achieved with the polymers, coupled with prolonged elimination period of the active from the body achieved by using probenecid as an adjuvant, provides the route through which once a day administration of a betalactam antibiotic like cephalexin or cefaclor composition is achieved.

Thus the object of the present invention is to provide a long acting pharmaceutical composition of a betalactam antibiotic such as cephalexin, cefaclor or their pharmaceutically acceptable hydrates, salts or esters in a modified release matrix formulation. These betalactam antibiotics, the drugs that are primarily excreted through active tubular secretion, may be optionally coupled with probenecid in controlled release or immediate release matrix, which is designed such that the resulting composition maintains the blood level of the active compound such that it is suitable for once or twice daily administration.

### SUMMARY OF THE INVENTION

Accordingly the present invention relates to a pharmaceutical composition for controlled release of an active ingredient, said composition comprising a controlled release matrix comprising a betalactam antibiotic as the active ingredient, and a mixture of hydrophilic polymers, said hydrophilic polymers being selected from the group consisting of at least one sodium alginate and at least one xanthan gum; and optionally probenecid as either immediate release or controlled release part, wherein said composition comprises about 30% to about 90% by weight of active ingredient and about 1% to about 25% by weight of hydrophilic polymers comprising from about 0.1% to about 20% by weight of sodium alginate and about 0.1% to about 20% by weight of xanthan gum.

The composition optionally also contains one or more water soluble and/or water dispersible diluents, wherein the quantities of the hydrophilic polymers and water soluble and/or water dispersible diluents are such that the therapeutically effective active ingredient is released at a rate suitable for once or twice daily administration of the pharmaceutical composition. Inclusion of probenecid allows reduction in the amount of active incorporated in the hydrophilic polymer matrix but still provides the desired once a day profile.

The betalactam antibiotic is selected from cephalexin, cefaclor or their pharmaceutically acceptable hydrates, salts or esters.

The modified release matrix formulation not containing probenecid prepared according to present invention may be administered once or twice daily. For example, the effective therapeutic dose of the active that can be administered by compositions of present invention include 500 mg active twice daily or 1.5 g active once daily.

The modified release matrix formulation containing probenecid prepared according to present invention may be administered once daily. For example, the effective therapeutic dose of the active that can be administered by compositions of present invention include 1.0 g active and 1.0 g probenecid once daily.

### DETAILED DESCRIPTION OF THE INVENTION

The composition of this invention is in the form of a matrix tablet comprising the active ingredient, hydrophilic polymers, water soluble and/or water dispersible diluents, pharmaceutically acceptable tablet excipients, and antibiotic adjuvant if any, for controlling the release of active ingredients.

According to the present invention, the active ingredient is a betalactam antibiotic such as cephalexin, cefaclor or their pharmaceutically acceptable hydrates, salts or esters in a controlled release matrix. The cephalexin, cefaclor or their pharmaceutically acceptable hydrates, salts or esters may be present in an amount from about 30% to about 90% by weight of the controlled release matrix.

Further, the cephalexin, cefaclor or their pharmaceutically acceptable hydrates, salts or esters may be present in an amount from 100 mg to 2000 mg.

Examples of other cephalosporin antibiotics which may be used include cefuroxime, cefamandole, cefoxitin, cephalothin, moxalactam, cephapirin, ceffizoxime, cefonicid and, pharmaceutically acceptable hydrates, salts or esters thereof. Examples of other betalactam antibiotics, which may be used are amoxicillin, ampicillin, and cloxacillin.

According to the present invention the probenecid is used as an antibiotic adjuvant for reducing the elimination rate and increasing the half-life of the therapeutically active ingredient. Inclusion of probenecid allows reduction in the amount of active incorporated in the hydrophilic polymer matrix but still provides the desired once a day profile.

According to the present invention the pharmaceutical composition contains a mixture of hydrophilic polymers selected from the group consisting of sodium alginate and xanthan gum.

Xanthan gum when used as matrix forming agent in sustained release tablets, releases the drug slightly faster in acidic media, due to more rapid initial surface erosion than at higher pH. After hydration of the gum the drug release is essentially pH independent.

Sodium alginate is a water soluble salt of alginic acid. Sodium alginate is insoluble below pH 3 and soluble above pH 3. The matrix formed by sodium alginate releases the drug slowly below pH 3 and shows a faster release rate above pH 3. Thus, when it is used along with xanthan gum to form the matrix it reduces the initial bursting effect and in the later stages, acts as channeling agent to increase the release rate of the drug. These two polymers when used in appropriate concentrations provide the desired release profile, when the delivery system travels through the GIT, having varying pH gradients. Surprisingly, the polymers in appropriate combinations are not only effective compared with commonly used polymers, but works at low concentrations.

According to the present invention, the pharmaceutical composition contains a mixture of hydrophilic polymers of different viscosity grades selected from the group consisting of sodium alginate and xanthan gum. For the purpose of this patent application, sodium alginate may be characterized by their viscosities in a 1% w/w aqueous solution as low viscosity 30 (about 75 to about 150 cPs), medium viscosity (about 200 to about 400 cPs) and high viscosity (about 600 to about 1000 cPs); and xanthan gum may be characterized as low viscosity (about 600 to about 1500 cPs), medium viscosity (about 1550 to about 1850 cPs) and high viscosity (greater than about 1900 cPs).

The different viscosity grade sodium alginate polymers that may be used in the present invention include, for example polymers available under the brand names Protanal LF 240 D™, Protanal SF 120RB™; available from FMC BioPolymers, Norway and Keltone HVCR™ available from Kelco.

The xanthan gum polymers that may be used in the present invention include, for example medium or high viscosity grade polymers available from Jungbunzlauer, Austria and Rhodigel™, medium viscosity grade polymer available from Rhodia, USA. Low viscosity grade xanthan gum can also be used but in order to utilize minimum possible concentrations of the polymer to achieve the desired profiles, without comprising on the integrity of the matrix, medium or high viscosity grades are preferred.

In a preferred embodiment of the present invention, the pharmaceutical composition comprises from about 30% to about 90% by weight of cephalexin, cefaclor or their pharmaceutically acceptable hydrates, salts or esters and about 1% to about 25% by weight of hydrophilic polymers comprising of sodium alginate in an amount from about 0.1% to about 20% by weight and xanthan gum in an amount from about 0.1% to about 20% by weight of controlled release matrix.

In a more preferred embodiment of the present invention, the pharmaceutical composition comprises from about 30% to about 90% by weight of cephalexin, cefaclor or their pharmaceutically acceptable hydrates, salts or esters and about 1% to about 20% by weight of hydrophilic polymers comprising of a low or a medium viscosity grade sodium alginate in an amount from about 0.1% to about 15% by weight and a medium or a high viscosity grade xanthan gum in an amount from about 0.1% to about 15% by weight of controlled release matrix.

In one more preferred embodiment of present invention, the pharmaceutical composition comprises from about 30% to about 90% by weight of cephalexin, cefaclor or their pharmaceutically acceptable hydrates, salts or esters and about 1% to about 18% by weight of hydrophilic polymers comprising of a low or a medium viscosity grade sodium alginate in an amount from about 1.0% to about 10.0% by weight and a medium or a high viscosity grade xanthan gum in an amount from about 1.0% to about 10.0% by weight of controlled release matrix.

Probenecid when incorporated in the controlled release or immediate release matrix is present in an amount of from about 250 mg to about 1000 mg.

The inclusion of probenecid in pharmaceutical compositions allows lowering of amount of active used. For example, therapeutically effective dose of the active that can be administered by the compositions of the present invention containing probenecid include 1.0 g active and 1.0 g probenecid once daily, and therapeutically effective dose of the active that can be administered by the compositions of the present invention not containing probenecid include 1.5 g active once daily.

The immediate release probenecid part of the present invention contains disintegrating agent at a concentration in the range of about 2% to about 9% by weight of immediate release part. Preferably the disintegrating agent is sodium starch glycolate.

The composition may contain one or more of pharmaceutically acceptable excipients in an amount of about 1% to about 30% by weight of the total weight of the composition. These excipients may be water soluble or water dispersible. Examples of water soluble diluents that may be used in the present invention include lactose, mannitol, glucose, sorbitol, maltose, dextrates, dextrins and the like. Water dispersible diluent refers to insoluble pharmaceutical excipients, which disperse readily in water such as microcrystalline cellulose, starch, pre-gelatinized starch, magnesium aluminum silicates and the like. In one preferred embodiment, the water soluble diluent is lactose in amounts from about 4% to about 20% by weight. In another preferred embodiment, the water dispersible diluent is microcrystalline cellulose present in amount from about 4% to about 15% by weight.

The composition may also contain tablet lubricants, at a concentration in the range of about 0.2% to 5% by weight either alone or in combination of total weight of the composition. The lubricants that may be used include talc, stearic acid, magnesium stearate, colloidal silicon dioxide, calcium stearate, zinc stearate, hydrogenated vegetable oil and the like. Preferably the lubricant is selected from talc, stearic acid, magnesium stearate and colloidal silicon dioxide.

The pharmaceutical composition of the present invention may be prepared by procedures well known to formulation chemists. The method of manufacturing can affect the release characteristics of the composition. The active or their pharmaceutically acceptable hydrates, salts or esters; the hydrophilic polymer of which at least one is sodium alginate and at least one is xanthan gum; one or more water soluble or water dispersible diluents are either mixed together with lubricants and the blend is directly compressed into tablets or are granulated by compaction followed by sieving and the granules obtained are compressed into tablets. The active ingredient can be given as controlled release tablets for once or twice a day administration or as controlled release tablet along with separate probenecid tablets as a combipack to be administered simultaneously or coupled with probenecid into a single monolithic or bilayered tablets for once a day administration. The fines incorporated in the blend of active granules form about 12% to about 30% by weight of controlled release part, preferably from about 12% to about 20%.

For the purposes of this patent application, fines denote the particles having size less than 250 microns.

The above-mentioned process has the advantage over its granulation by aqueous or non-aqueous vehicle used conventionally. The drugs cephalexin and cefaclor, which are sensitive to moisture and heat, can be effectively processed without any difficulty. The polymers used in the composition of present invention, xanthan gum and sodium alginate also are unstable above 60° C. and 70° C. respectively. As the process is devoid of use of any solvents the potential problem of limiting the residual organic solvent is eliminated.

The controlled release matrix formulation of the present invention is not a mere admixture but has properties different from the sum total of the properties of its ingredients. The probenecid part of the composition is prepared by mixing probenecid and one or more water soluble or water dispersible diluents together with lubricants and the blend is granulated by compaction followed by sieving and the granules obtained are lubricated and compressed into a tablet. The fines incorporated in the blend form about 10% to about 30% by weight of immediate release part, preferably from about 10% to about 20%.

The modified release matrix formulation not containing probenecid prepared according to present invention may be administered once or twice daily. For example, the effective therapeutic dose of the active that can be administered by compositions of present invention include 500 mg active twice daily or 1.5 g active once daily.

The modified release matrix formulation containing probenecid prepared according to present invention may be administered once daily. For example, the effective therapeutic dose of the active that can be administered by compositions of present invention include 1.0 g active and 1.0 g probenecid once daily.

The present invention is illustrated by the following examples. Examples are not intended to be limiting to the scope of the invention.

### EXAMPLES

In all the examples where viscosity grade of xanthan gum is not mentioned in the respective tables, medium viscosity grade xanthan gum manufactured by Jungbunzlauer, Austria has been employed.

Tablets Without Probenecid

Cefaclor or cephalexin, hydrophilic polymers, lactose and microcrystalline cellulose were screened through a 30 mesh sieve and mixed with magnesium stearate. The blend was compacted and the slugs obtained were milled to form granules. The sized granules were blended with the fines and the remaining lubricant and further compressed into tablets.

For Example 1 to Example 7, the tablets were tested for cefaclor or cephalexin release in 900 ml of 0.1 N hydrochloric acid for 1 hr, after which the dissolution media was changed to pH 6.8 phosphate buffer 900 ml. The tablets were placed into a 40 mesh basket (USP apparatus type-I) and were rotated at 100 rpm. For the examples containing cefaclor, the dissolution medium (pH 6.8 phosphate buffer) was replaced with fresh medium every hour thereafter. For the examples containing cephalexin only aliquots and not all of the pH 6.8 phosphate buffer were withdrawn and replaced with fresh medium at each interval.

### Example 1

| Ingredients | Weight (mg/tablet) | % w/w |
|---|---|---|
| Cefaclor | 530.50 | 78.01 |
| Sodium alginate (Protanal LF 240 D) | 6.80 | 1.00 |
| Xanthan gum | 3.40 | 0.50 |
| Lactose monohydrate | 78.20 | 11.50 |
| Microcrystalline cellulose | 54.33 | 7.99 |
| Magnesium Stearate | 6.80 | 1.00 |

| Time (hour) | Percent Cefaclor Released (%) |
|---|---|
| 1 | 39.20 |
| 2 | 62.30 |
| 3 | 79.00 |
| 4 | 91.10 |

### Example 2

| Ingredients | Weight (mg/tablet) | % w/w |
|---|---|---|
| Cefaclor | 530.50 | 88.41 |
| Sodium alginate (Protanal LF 240 D) | 24.00 | 4.00 |
| Xanthan gum | 6.00 | 1.00 |
| (high viscosity grade, Jungbunzlauer) | | |
| Lactose monohydrate | 33.50 | 5.59 |
| Magnesium Stearate | 6.00 | 1.00 |

| Time (hour) | Percent Cefaclor Released (%) |
|---|---|
| 1 | 30.10 |
| 2 | 54.50 |
| 3 | 72.70 |
| 4 | 85.80 |

### Example 3

| Ingredients | Weight (mg/tablet) | % w/w |
|---|---|---|
| Cefaclor | 519.19 | 72.11 |
| Sodium alginate (Protanal LF 240 D) | 36.00 | 5.00 |
| Xanthan gum | 14.40 | 2.00 |
| Lactose monohydrate | 57.60 | 8.00 |
| Microcrystalline cellulose | 87.40 | 12.14 |
| Magnesium Stearate | 5.40 | 0.75 |

| Time (hour) | Percent Cefaclor Released (%) |
|---|---|
| 1 | 40.50 |
| 2 | 62.70 |
| 3 | 80.90 |
| 4 | 92.10 |

### Example 4

| Ingredients | Weight (mg/tablet) | % w/w |
|---|---|---|
| Cefaclor | 530.50 | 85.84 |
| Sodium alginate (Protanal LF 240 D) | 24.00 | 3.88 |
| Xanthan gum | 6.00 | 0.97 |
| Lactose monohydrate | 48.50 | 7.84 |
| Magnesium Stearate | 9.00 | 1.45 |

| Time (hour) | Percent Cefaclor Released (%) |
|---|---|
| 1 | 34.10 |
| 2 | 58.30 |
| 3 | 79.60 |
| 4 | 91.30 |

### Example 5

| Ingredients | Weight (mg/tablet) | % w/w |
|---|---|---|
| Cefaclor | 530.50 | 78.00 |
| Sodium alginate (Protanal LF 240 D) | 20.40 | 3.00 |
| Xanthan gum | 13.60 | 2.00 |
| Lactose monohydrate | 54.40 | 8.00 |
| Microcrystalline cellulose | 54.40 | 8.00 |
| Magnesium Stearate | 6.80 | 1.00 |

| Time (hour) | Percent Cefaclor Released (%) |
|---|---|
| 1 | 40.10 |
| 2 | 54.30 |
| 3 | 68.30 |
| 4 | 88.00 |

### Example 6

| Ingredients | Weight (mg/tablet) | % w/w |
|---|---|---|
| Cephalexin | 519.21 | 76.35 |
| Sodium alginate (Protanal LF 240 D) | 34.00 | 5.00 |
| Xanthan gum | 13.60 | 2.00 |
| Lactose monohydrate | 54.40 | 8.00 |
| Microcrystalline cellulose | 52.02 | 7.65 |
| Magnesium Stearate | 6.80 | 1.00 |

| Time (hour) | Percent Cephalexin Released (%) |
|---|---|
| 1 | 33.40 |
| 2 | 50.60 |
| 3 | 66.30 |
| 4 | 79.00 |
| 5 | 92.30 |

### Example 7

| Ingredients | Weight (mg/tablet) | % w/w |
|---|---|---|
| Cefaclor | 518.67 | 74.09 |
| Sodium alginate (Keltone HVCR) | 21.00 | 3.00 |
| Xanthan gum | 7.00 | 1.00 |
| Lactose monohydrate | 139.33 | 19.90 |
| Magnesium Stearate | 14.00 | 2.00 |

| Time (hour) | Percent Cefaclor Released (%) |
|---|---|
| 1 | 30.80 |
| 2 | 55.30 |
| 3 | 75.00 |
| 4 | 88.00 |

For Example 8 to Example 10, the tablets were tested for cephalexin release in 900 ml of 0.1 N hydrochloric acid for 2 hr, after which the dissolution media was changed to pH 6.8 phosphate buffer 900 ml. The tablets were placed into a 40 mesh basket (USP apparatus type-I) and were rotated at 100 rpm.

### Example 8

| Ingredients | Weight (mg/tablet) | % w/w |
|---|---|---|
| Cephalexin | 812.92 | 75.27 |
| Sodium alginate (Protanal LF 240 D) | 86.40 | 8.00 |
| Xanthan gum (Rhodigel) | 64.80 | 6.00 |
| Lactose monohydrate | 107.45 | 9.95 |
| Magnesium Stearate | 8.43 | 0.78 |
| Weight | 1080 | 100 |

| Time (hour) | Percent Cephalexin Released (%) |
|---|---|
| 1 | 25.80 |
| 2 | 37.10 |
| 3 | 46.20 |
| 4 | 48.80 |
| 6 | 68.00 |
| 8 | 88.80 |
| 10 | 102.40 |

### Example 9

| Ingredients | Weight (mg/tablet) | % w/w |
|---|---|---|
| Cephalexin | 812.92 | 75.27 |
| Sodium alginate (Protanal LF 240 D) | 108.00 | 10.00 |
| Xanthan gum | 64.80 | 6.00 |
| Lactose monohydrate | 83.48 | 7.73 |
| Magnesium Stearate | 10.80 | 1.00 |
| Weight | 1080 | 100 |

| Time (hour) | Percent Cephalexin Released (%) |
|---|---|
| 1 | 26.60 |
| 2 | 40.00 |
| 3 | 50.50 |
| 4 | 57.90 |
| 6 | 84.30 |
| 8 | 93.50 |
| 10 | 97.80 |
| 12 | 103.40 |

### Example 10

| Ingredients | Weight (mg/tablet) | % w/w |
|---|---|---|
| Cephalexin | 812.92 | 75.27 |
| Sodium alginate (Protanal SF 120 RB) | 86.40 | 8.00 |
| Xanthan gum | 75.60 | 7.00 |
| Lactose monohydrate | 94.28 | 8.73 |
| Magnesium Stearate | 10.80 | 1.00 |
| Weight | 1080 | 100 |

| Time (hour) | Percent Cephalexin Released (%) |
|---|---|
| 1 | 22.30 |
| 2 | 35.90 |
| 3 | 45.20 |
| 4 | 45.70 |
| 6 | 54.60 |
| 8 | 65.30 |
| 10 | 79.30 |
| 12 | 92.50 |
| 14 | 105.10 |

Tablets With Probenecid

### Example 11

Cephalexin, hydrophilic polymers and mannitol were screened through a 30 mesh sieve and mixed with the magnesium stearate. The blend was compacted and the slugs obtained were milled to obtain granules. The sized granules were blended with fines and the remaining lubricant.

Probenecid, starch, sodium starch glycolate, were screened through 30 mesh sieve. The blend was compacted and the slugs obtained were again milled to obtain granules. The sized granules were mixed with remaining sodium starch glycolate and magnesium stearate and fines, followed by compression of cephalexin granules on the pre-compressed probenecid granules.

The tablets were tested for cephalexin release in 900 ml of 0.1 N hydrochloric acid for 2 hrs after which the dissolution media was changed to pH 6.8 phosphate buffer 900 ml. The tablets were placed into a 40 mesh basket (USP apparatus type I) and were rotated at 100 rpm. Further, fresh tablets were analysed for probenecid release using 900 ml of pH 7.5 simulated intestinal fluid without pancreatin, USP apparatus type II at 50 rpm.

| Ingredients | Weight (mg) | % w/w |
|---|---|---|
| Controlled Release Part | | |
| Cephalexin | 526.21 | 72.58 |
| Sodium alginate (Protanal LF 240 D) | 101.57 | 14.00 |
| Xanthan gum | 50.75 | 7.00 |
| Mannitol | 39.51 | 5.45 |
| Magnesium Sterate | 6.96 | 0.96 |
| Weight | 725.00 | 100.00 |
| Immediate Release Part | | |
| Probenecid | 500.00 | 79.36 |
| Microcrystalline Cellulose | 95.50 | 15.15 |
| Sodium Starch Glycolate | 31.50 | 5.00 |
| Magnesium Sterate | 3.00 | 0.47 |
| Weight | 630.00 | 100.00 |

| Time (hour) | Percent Cephalexin Released (%) |
|---|---|
| 1 | 28.00 |
| 2 | 45.40 |
| 3 | 57.50 |
| 4 | 61.80 |
| 6 | 70.20 |
| 8 | 80.60 |
| 10 | 92.70 |
| 12 | 101.60 |
| Time (min) | Percent Probenecid Released (%) |
| 10 | 98.80 |
| 20 | 102.20 |
| 30 | 103.30 |

### Example 12

Cephalexin, hydrophilic polymers and microcrystalline cellulose were screened through 30 mesh sieve and mixed together with magnesium stearate. The blend was compacted and the slugs obtained were milled to obtain granules. The sized granules then blended with the fines and the extra granular lubricant.

Probenecid, lactose, sodium starch glycolate were screened through 30 mesh sieve. The blend was compacted and slugs obtained were milled to obtain granules. The sized granules were mixed with remaining sodium starch glycolate, magnesium stearate and fines followed by compression of cephalexin granules on pre-compressed probenecid granules.

The tablets were tested for dissolution in 900 ml of 0.1 N hydrochloric acid for 2 hrs after which the dissolution media was changed to pH 6.8 phosphate buffer 900 ml, using 40 mesh basket (USP apparatus type I) and were rotated at 100 rpm. Further fresh samples were analysed for probenecid release using 900 ml of pH 7.5 simulated intestinal fluid without pancreatin, USP apparatus Type II at 50 rpm.

| | Weight | |
|---|---|---|
| Ingredients | (mg/tablet) | % w/w |
| Controlled Release Part | | |
| Cephalexin | 539.95 | 74.47 |
| Sodium alginate (Protanal LF 240 D) | 50.75 | 7.00 |
| Xanthan gum | 43.50 | 6.00 |
| (high viscosity grade, Jungbunzlauer) | | |
| Microcrystalline cellulose | 85.26 | 11.76 |
| Magnesium Sterate | 5.54 | 0.76 |
| Weight | 725.00 | 100.00 |
| Immediate Release Part | | |
| Probenecid | 500.00 | 79.36 |
| Lactose | 102.00 | 16.19 |
| Sodium Starch Glycolate | 25.00 | 3.96 |
| Magnesium Sterate | 3.00 | 0.47 |
| Weight | 630.00 | 100.00 |

| Time (hour) | Percent Cephalexin Released (%) |
|---|---|
| 1 | 27.60 |
| 2 | 43.00 |
| 3 | 52.40 |
| 4 | 56.10 |
| 6 | 68.50 |
| 8 | 80.60 |
| 10 | 95.90 |
| Time (min) | Percent Probenecid Released (%) |
| 10 | 80.30 |
| 20 | 95.40 |
| 30 | 99.30 |

### Example 13

Cephalexin, hydrophilic polymers and microcrystalline cellulose were screened through 30 mesh sieve and mixed together with magnesium stearate. The blend was compacted and the slugs obtained were milled to obtain granules. The sized granules then blended with the fines and the extra granular lubricant.

Probenecid, lactose, sodium starch glycolate were screened through 30 mesh sieve. The blend was compacted and slugs obtained were milled to obtain granules. The sized granules were mixed with remaining sodium starch glycolate, magnesium stearate and fines followed by compression of cephalexin granules on pre-compressed probenecid granules.

The tablets were tested for dissolution in 900 ml of 0.1 N hydrochloric acid for 2 hrs after which the dissolution media was changed to pH 6.8 phosphate buffer 900 ml, using 40 mesh basket (USP apparatus type I) and were rotated at 100 rpm. Further fresh samples were analysed for probenecid release using 900 ml of pH 7.5 simulated intestinal fluid without pancreatin, USP apparatus Type II at 50 rpm.

| | Weight | |
|---|---|---|
| Ingredients | (mg/tablet) | % w/w |
| Controlled Release Part | | |
| Cephalexin | 539.95 | 74.47 |
| Sodium alginate (Protanal SF 120 RB) | 43.50 | 6.00 |
| Xanthan gum | 43.50 | 6.00 |
| Microcrystalline cellulose | 92.51 | 12.77 |
| Magnesium Sterate | 5.54 | 0.76 |
| Weight | 725.00 | 100.00 |
| Immediate Release Part | | |
| Probenecid | 500.00 | 79.36 |
| Lactose | 102.00 | 16.19 |
| Sodium Starch Glycolate | 25.00 | 3.96 |
| Magnesium Sterate | 3.00 | 0.47 |
| Weight | 630.00 | 100.00 |

| Time (hour) | Percent Cephalexin Released (%) |
|---|---|
| 1 | 26.50 |
| 2 | 40.10 |
| 3 | 55.40 |
| 4 | 58.20 |
| 6 | 68.50 |
| 8 | 84.00 |
| 10 | 95.90 |
| Time (min) | Percent Probenecid Released (%) |
| 10 | 85.43 |
| 20 | 95.21 |
| 30 | 99.13 |

### Example 14

Cephalexin, probenecid, hydrophilic polymers and microcrystalline cellulose were screened through 30 mesh screen and mixed with Magnesium stearate. The blend was compacted and the slugs obtained were milled to obtain the granules. The sized granules were blended with fines and the remaining lubricant and further compressed into tablets.

The tablets were tested for dissolution in 900 ml of 0.1 N hydrochloric acid for 2 hrs after which the dissolution media was changed to pH 6.8 phosphate buffer 900 ml, using 40 mesh basket (USP apparatus type I) and were rotated at 100 rpm. Further fresh samples were analysed for probenecid release using 900 ml of pH 7.5 simulated intestinal fluid without pancreatin, USP apparatus Type II at 50 rpm.

| | Weight | |
|---|---|---|
| Ingredients | (mg/tablet) | % w/w |
| Cephalexin | 539.95 | 40.77 |
| Probenecid | 500.00 | 37.73 |
| Sodium alginate (Protanal LF 240 D) | 79.30 | 5.90 |
| Xanthan gum | 132.50 | 10.00 |
| Microcrystalline cellulose | 60.00 | 4.52 |
| Magnesium Sterate | 13.25 | 1.00 |
| Weight | 1325.00 | 100.00 |

| Time | Percent Cephalexin | Percent Probenecid |
|---|---|---|
| (hour) | Released (%) | Released (%) |
| 1 | 26.10 | 45.20 |
| 2 | 40.20 | 70.10 |
| 3 | 55.10 | 88.10 |
| 4 | 63.40 | 100.40 |
| 6 | 72.10 | |
| 8 | 81.40 | |
| 10 | 92.90 | |
| 12 | 100.80 | |

### Example 15

Cefaclor, hydrophilic polymers and mannitol were screened through a 30 mesh sieve and mixed with the magnesium stearate. The blend was compacted and the slugs obtained were milled to obtain granules. The sized granules were blended with fines and the remaining lubricant.

Probenecid, starch, sodium starch glycolate, were screened through 30 mesh sieve. The blend was compacted and the slug obtained were again milled to obtain granules. The sized granules were mixed with remaining sodium starch glycolate and magnesium stearate and fines, followed by compression of cefaclor granules on the pre-compressed probenecid granules.

The tablets were tested for cefaclor release in 900 ml of 0.1 N hydrochloric acid for 2 hrs after which the dissolution media was changed to pH-6.8 phosphate buffer 900 ml. The tablets were placed into a 40 mesh basket (USP apparatus type-I) and were rotated at 100 rpm. The dissolution medium (pH 6.8 phosphate buffer) was replaced by fresh medium every hour. Further fresh tablet were analysed for probenecid release using 900 ml of pH 7.5 simulated intestinal fluid without pancreatin, USP apparatus type II at 50 rpm.

| | Weight | |
|---|---|---|
| Ingredients | (mg/tablet) | % w/w |
| Controlled Release Part | | |
| Cefaclor | 527.40 | 75.34 |
| Sodium alginate (Protanal LF 240 D) | 84.00 | 12.00 |
| Xanthan gum (Rhodigel) | 35.00 | 5.00 |
| Mannitol | 43.10 | 6.15 |
| Magnesium Sterate | 10.50 | 1.50 |
| Weight | 700.00 | 100.00 |
| Immediate Release Part | | |
| Probenecid | 500.00 | 79.36 |
| Starch | 70.61 | 11.20 |
| Sodium Starch Glycolate | 50.40 | 8.00 |
| Magnesium Sterate | 9.00 | 1.42 |
| Weight | 630.00 | 100.00 |

| Time (hour) | Percent Cefaclor Released (%) |
|---|---|
| 1 | 29.00 |
| 2 | 44.60 |
| 3 | 51.20 |
| 4 | 57.40 |
| 6 | 68.20 |
| 8 | 79.90 |
| 10 | 92.80 |
| 12 | 100.00 |
| Time (min) | Percent Probenecid Released (%) |
| 10 | 95.40 |
| 20 | 99.20 |
| 30 | 100.20 |

### Example 16

Cefaclor, probenecid, hydrophilic polymers, mannitol and microcrystalline cellulose were screened through a 30 mesh sieve and mixed with the magnesium stearate. The blend was compacted and the slug obtained were milled to obtain granules. The sized granules were blended with fines and the remaining lubricant and further compressed into tablets.

The tablets were tested for cefaclor release in 900 ml of 0.1 N hydrochloric acid for 2 hrs after which the dissolution media was changed to pH 6.8 phosphate buffer 900 ml, using 40 mesh (USP apparatus type I) and were rotated at 100 rpm. The dissolution medium (pH 6.8 phosphate buffer) was replaced by fresh medium every hour. The results are given in table 4. Further fresh samples were analysed for probenecid release using 900 ml of pH 7.5 simulated intestinal fluid without pancreatin USP apparatus Type II at 50 rpm.

| | Weight | |
|---|---|---|
| Ingredients | (mg/tablet) | % w/w |
| Cefaclor | 527.40 | 38.35 |
| Probenecid | 500.00 | 36.36 |
| Sodium alginate (Protanal LF 240 D) | 68.00 | 9.00 |
| Xanthan gum (Rhodigel) | 58.29 | 4.23 |
| Mannitol | 123.50 | 9.00 |
| Microcrystalline cellulose | 82.50 | 6.00 |
| Magnesium Sterate | 15.00 | 1.09 |
| Weight | 1375.00 | 100.00 |

| Time | Percent Cefaclor | Percent Probenecid |
|---|---|---|
| (hour) | Released (%) | Released (%) |
| 1 | 28.00 | 47.10 |
| 2 | 40.60 | 71.20 |
| 3 | 55.20 | 85.40 |
| 4 | 60.40 | 100.20 |
| 6 | 67.80 | |
| 8 | 79.40 | |
| 10 | 91.40 | |
| 12 | 100.10 | |

### BRIEF DESCRIPTION OF THE DRAWINGS

The dissolution profile of the active and the release over time of the active in-vivo is illustration in greater details with reference to graphical representation shown in the accompanying drawings, in which

FIG. 1 is a graph illustrating the dissolution profile of the active when sodium alginate alone or xanthan gum alone or combination of sodium alginate and xanthan gum is used.

FIG. 2 is a graph illustrating the release over time of cefaclor into the bloodstream for a composition of the present invention and a reference formulation.

FIG. 3 is a graph illustrating the release over time of cephalexin into the bloodstream for a modified release composition of the present invention containing probenecid and conventional cephalexin composition.

FIG. 4 is a graph illustrating the release over time of cephalexin into the bloodstream for a modified release composition of the present invention without probenecid and conventional cephalexin composition.

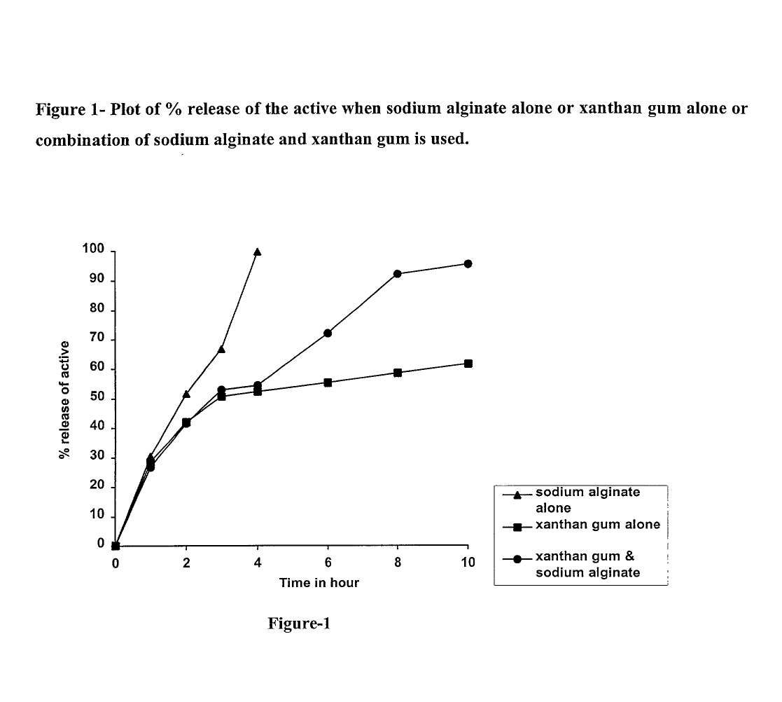

We have conducted trials using xanthan gum and sodium alginate alone, and in combination with each other. The results obtained when sodium alginate alone in 16% concentration, xanthan gum alone in 16% concentration and combination of sodium alginate and xanthan gum in 7 and 6% concentration, respectively, is used, are summarized in Table 1 and graphically represented in FIG. 1. Xanthan gum in varying concentrations gave a faster drug release, initially due to rapid surface erosion. The drug release retarded at a later stage due to hydration. Sodium alginate gave slower initial release, however, the release could not be controlled at a later stage due to higher solubility at higher pH. The combination of xanthan gum and sodium alginate forms a integrated matrix, incorporation of sodium alginate in the matrix results in desired retarded release initially and acts as a channeling agent, to increase the release rate at a later stage, thus, compensating the further retardation due to hydration of xanthan gum. The combination of these two polymers compliment each other such that, it overcomes the deficiencies associated with their use, when used alone.

TABLE 1
% Release of the active

| | Conventional | Sodium | Xanthan | |
|---|---|---|---|---|
| Duration | (without | alginate | gum | Xanthan gum + |
| in hr | polymers) | alone | alone | Sodium alginate |
| 1 | 100 | 30.4 | 28.5 | 26.8 |
| 2 | | 51.7 | 42.0 | 41.5 |
| 3 | | 66.7 | 50.7 | 53.0 |
| 4 | | 99.7 | 52.4 | 54.4 |
| 6 | | | 55.2 | 71.9 |
| 8 | | | 58.5 | 92.0 |
| 10 | | | 61.5 | 95.4 |

A study was conducted for bioequivalence between a cefaclor test formulation (T) in accordance with the composition of this invention and a reference product (R) already being marketed, Ceclor™ CD (Eli Lilly), both having a cefaclor content equivalent to 500 mg. Eight healthy male volunteers were selected for a randomized, two way crossover bioequivalence study in which each volunteer was administered a dose of the drug with 180 ml of water. The volunteers fasted overnight before taking the drug.

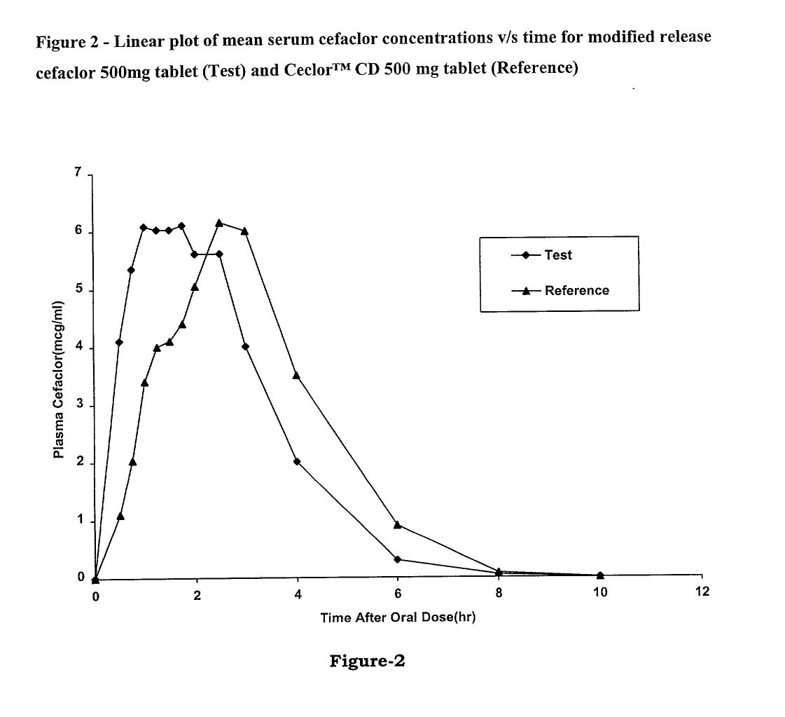

FIG. 2 shows a plot of blood level concentration of cefaclor for the reference product (R) in comparison with that of present invention (T). The test formulation was found to be bioequivalent to the reference product.

In vivo study for bioavailability was conducted for comparison between conventional cephalexin and long acting modified release matrix formulation containing probenecid, one dose of 2 tablets×(500 mg cephalexin+500 mg probenecid), prepared according to present invention. Eight healthy male volunteers were selected for the study in which each volunteer was administered a dose of the drug with 180 ml of water. The volunteers fasted overnight and had a standard breakfast before taking the drug. After administration of 500 mg cephalexin conventional dosage form, the blood levels are achieved within 1 hour and detectable blood levels are present for 6 hours, whereas long acting modified release formulation containing probenecid according to present invention gave the desired blood levels up to 18 to 20 hours, clearly indicating that it can be used as once daily composition.

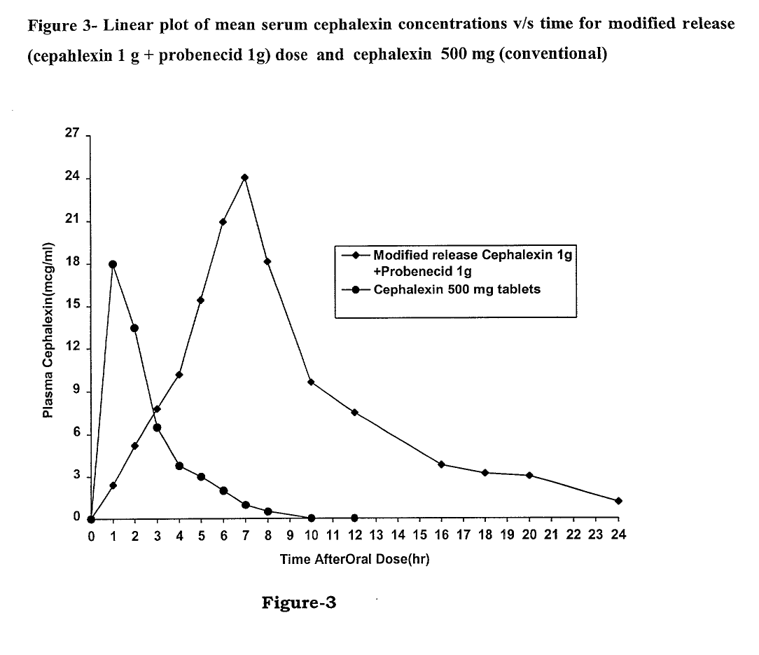

FIG. 3 shows a plot of blood level concentrations of modified release composition, one dose of 2 tablets×(cephalexin 500 mg+probenecid 500 mg) V/s cephalexin 500 mg conventional tablet.

The bioavailability study was also conducted for comparison between conventional cephalexin and long acting modified release matrix formulation without probenecid, one dose of 2 tablets×(750 mg cephalexin), prepared according to present invention. Eight healthy male volunteers were selected for the study in which each volunteer was administered a dose of the drug with 180 ml of water. The volunteers had a standard breakfast before taking the drug. The desired blood levels up to 18 to 20 hours were achieved with compositions without probenecid prepared according to the invention, clearly indicating that it can be used as once daily composition.

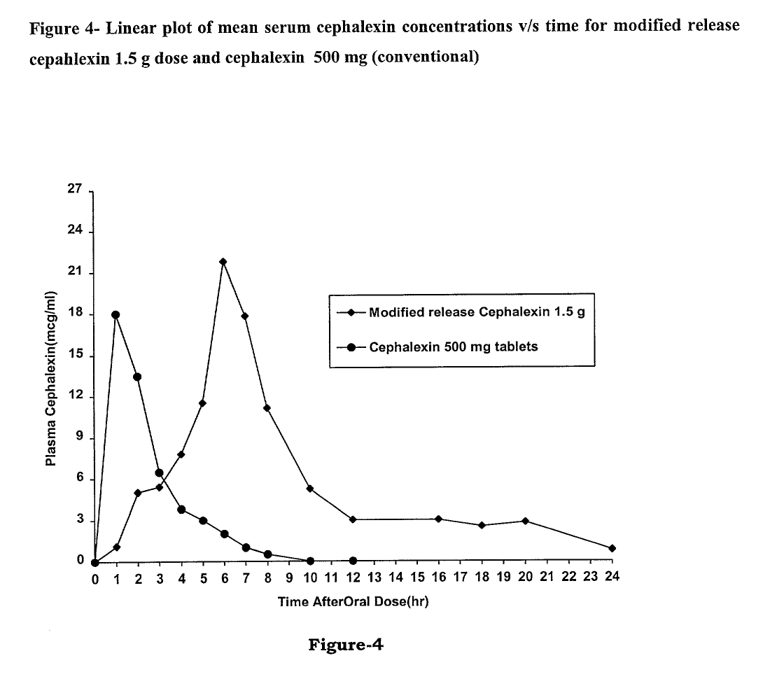

FIG. 4 shows a plot of blood level concentrations of modified release composition, one dose of 2 tablets×(cephalexin 750 mg) V/s cephalexin 500 mg conventional tablet.

1. A pharmaceutical composition for controlled release of an active ingredient, said composition comprising a controlled release matrix comprising a betalactam antibiotic or their pharmaceutically acceptable hydrates, salts or esters as the active ingredient, and a mixture of hydrophilic polymers, said hydrophilic polymers being selected from the group consisting of at least one sodium alginate and at least one xanthan gum; and optionally probenecid as either immediate release or controlled release part, wherein said composition comprises about 30% to about 90% by weight of active ingredient and about 1% to about 25% by weight of hydrophilic polymers comprising from about 0.1% to about 20% by weight of sodium alginate and about 0.1% to about 20% by weight of xanthan gum.

2. The composition as claimed in claim 1, wherein the betalactam antibiotic is selected from cephalexin, cefaclor or their pharmaceutically acceptable hydrates, salts or esters.

3. The composition as claimed in claim 1, wherein the active is released at a rate suitable for once daily or twice daily administration of the composition.

4. The composition as claimed in claim 1 in the form of a solid dosage.

5. The composition as claimed in claim 1, wherein the controlled release part of the pharmaceutical composition comprises from about 30% to about 90% by weight of active ingredient or their pharmaceutically acceptable hydrates, salts or esters and about 1% to about 20% by weight of hydrophilic polymers comprising of a low or a medium viscosity grade sodium alginate in an amount from about 0.1% to about 15% by weight and a medium or a high viscosity grade xanthan gum in an amount from about 0.1% to about 15% by weight.

6. The composition as claimed in claim 1, wherein the controlled release part of the pharmaceutical composition comprises from about 30% to about 90% by weight of active ingredient or their pharmaceutically acceptable hydrates, salts or esters and about 1% to about 18% by weight of hydrophilic polymers comprising of a low or a medium viscosity grade sodium alginate in an amount from about 1.0% to about 10.0% by weight and a medium or a high viscosity grade xanthan gum in an amount from about 1.0% to about 10.0% by weight.

7. The composition as claimed in claim 1, which further contains at least one water soluble or water dispersible diluent.

8. The composition as claimed in claim 7, wherein the water soluble or water dispersible diluent comprises about 1% to about 30% by weight of the composition.

9. The composition as claimed in claim 7, wherein the diluent is lactose.

10. The composition as claimed in claim 9, wherein the amount of lactose is from about 4% to about 20% by weight of the composition.

11. The composition as claimed in claim 7, wherein the diluent is microcrystalline cellulose.

12. The composition as claimed in claim 11, wherein the amount of microcrystalline cellulose is from about 4% to about 15% by weight of the composition.

13. The composition as claimed in claim 1 further comprising magnesium stearate, talc, colloidal silicon dioxide or mixtures thereof in amounts from about 0.2% to about 5% by weight each.

14. A composition as claimed in any preceding claim wherein a multidose contains 250 mg to 2 g active ingredient.

15. A composition as claimed in claims 1 to 13 in dosage unit form containing 100 to 1 g of active ingredient.

16. A process for the preparation of a pharmaceutical composition as claimed in claim 1, comprising mixing together the active ingredient and the hydrophilic polymers, and optionally probenecid together with at least one lubricant to form a blend, further compacting, sizing, blending and compressing into tablets.

17. A process for the preparation of a pharmaceutical composition as claimed in claim 1, comprising mixing together the active ingredient and the hydrophilic polymers together with at least one lubricant to form a blend, further compacting, sizing, blending and compressing into tablets along with immediate release probenecid part.

18. The composition as claimed in claim 1, wherein probenecid is present in an amount from about 250 mg to about 1000 mg.

19. The composition as claimed in claim 1, which further contains a disintegrating agent from about 2% to about 9% by weight of immediate release part.

20. The composition as claimed in claim 19 wherein disintegrating agent is sodium starch glycolate.

21. A process for the preparation of pharmaceutical composition as claimed in claim 1, comprising mixing together probenecid, diluent and disintegrating agent together, compacting, sizing and blending with lubricant and compressing the blend into tablets along with controlled release part of the active ingredient.

22. The composition as claimed in claim 1 wherein, the controlled release part in a hydrophilic matrix and immediate release probenecid part are compressed together into a tablet dosage form.

23. The composition as claimed in claim 1 wherein, the controlled release part in a hydrophilic matrix and immediate release probenecid part are compressed separately into tablets and packed in a way to be administered simultaneously.
